(19) United States
(12) Patent Application Publication
(10) Pub. No.: US 2024/0285282 A1
(43) Pub. Date: Aug. 29, 2024

(54) ENDOVASCULAR DECOMPRESSION OF ANUERYSMS

(71) Applicant: Covidien LP, Mansfield, MA (US)

(72) Inventors: Dragos A. Agafitei, Aliso Viejo, CA (US); Nina M. Agafitei, Irvine, CA (US)

(21) Appl. No.: 18/582,334

(22) Filed: Feb. 20, 2024

Related U.S. Application Data
(60) Provisional application No. 63/486,602, filed on Feb. 23, 2023.

(51) Int. Cl. A61B 17/12

(52) U.S. Cl. CPC A61B 17/12118; A61B 2217/005

(57) ABSTRACT

A medical system for treating aneurysms includes a medical device configured to direct fluid flow away from an aneurysm. The medical device includes a body configured to be positioned across a neck of an aneurysm. The body defines an opening configured to enable fluid flow out of the aneurysm through the neck when the body is positioned across the neck. The medical device further includes a valve configured to allow unidirectional flow of fluid through the opening from the aneurysm into a vessel when the body is positioned across the neck. A method for treating aneurysms includes positioning the body of the medical device across a neck of an aneurysm. The method further includes positioning a catheter in a vessel proximate the neck. The catheter defines a catheter lumen. The method further includes applying suction to the catheter lumen to remove fluid from the aneurysm through the valve.

This application claims the benefit of U.S. Provisional Application No. 63/486,602, filed on Feb. 23, 2023, and entitled, “ENDOVASCULAR DECOMPRESSION OF ANEURYSMS,” the entire content of which is incorporated herein by reference.

### TECHNICAL FIELD

This disclosure relates to medical devices configured to treat aneurysms.

### BACKGROUND

Aneurysms can occur when constant, high pressure applied to a blood vessel wall results in a thinning or weakening of the blood vessel wall. Some aneurysms form as bulge or balloon-like structures that protrude from the blood vessel wall and become weaker as they grow, increasing the risk of rupture over time.

### SUMMARY

This disclosure describes medical devices and systems configured to facilitate decompression (e.g., collapsing) of an aneurysm via aspiration and divert blood flow from the aneurysm. In examples described herein, a medical device includes a valve configured to allow unidirectional flow of fluid through an opening of the medical device from an aneurysm into a vessel, e.g., to enable blood to flow out of the aneurysm to decompress the aneurysm and also minimize or even prevent blood from flowing back into the aneurysm after decompression. For example, in some cases, a medical device is configured to be positioned across a neck of an aneurysm and a one-way valve included in the device is configured to direct fluid flow out of the aneurysm through the neck and away from the aneurysm into a vessel. The flow of fluid out of the aneurysm through the valve can be facilitated by application of suction force (referred to herein as aspiration) to facilitate relatively quick decompression of the aneurysm. By aspirating the blood from within the aneurysm, the aneurysm is able to quickly collapse, which may be advantageous in minimizing aneurysm rupture risk, reducing intercranial pressure, and improving overall healing.

In some examples, a medical device includes an expandable frame and polymer layer defining one or more flaps configured to open towards a central longitudinal axis of the frame to allow the unidirectional flow of fluid out of the aneurysm. In these examples, the one or more flaps define a one-way valve. In other examples, the medical device comprises a valve defined by at least two overlapping polymer layers that are configured to enable fluid flow through a space between the at least two polymer layers. The overlapping polymer layers can define a one-way valve. In still other examples, the medical device comprises an expandable mesh configured to occlude at least part of the neck of the aneurysm and a valve configured to allow unidirectional flow of fluid from the aneurysm into a vessel. The type of valve can include, but is not limited to, a spring check valve, a flap check valve or a plug check valve. Additionally, in some examples, the valve includes a membrane valve comprising a membrane configured to allow unidirectional flow of fluid from the aneurysm into a vessel.

Additionally, in some examples, the medical system further comprises a catheter configured to be positioned in the vessel and a suction source configured to apply a suction force to the catheter to remove fluid from the aneurysm through the valve. In addition, in some examples, a flow diverter and/or a distal embolic protection device (e.g., a filter and/or an occlusion device, such as a balloon catheter) can be used in conjunction with the valve.

This disclosure also describes examples of methods of positioning the body of the medical device across the neck of an aneurysm and methods of using the medical device.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic diagram illustrating an example of unidirectional flow of fluid from an aneurysm into a vessel.

FIG. 2 is a schematic diagram illustrating an example system that includes a medical device including a valve configured to allow unidirectional flow of fluid out of an aneurysm, the system further including a catheter configured to extend into the aneurysm through a neck of the aneurysm.

FIG. 3 is a schematic diagram of an example medical device including a valve configured to allow unidirectional flow of fluid out of an aneurysm.

FIG. 4 is another schematic diagram of another example medical device including a valve configured to allow unidirectional flow of fluid out of an aneurysm.

FIG. 5 is another schematic diagram of an example medical device including a valve configured to allow unidirectional flow of fluid out of an aneurysm.

FIG. 6 is a schematic diagram illustrating an example system including medical device including a valve configured to allow unidirectional flow of fluid out of an aneurysm, as well as an embolic protection device.

FIG. 7 is a schematic diagram illustrating an example system including medical device including a valve, an embolic protection device, and an aspiration catheter.

FIG. 8 is a schematic diagram illustrating an example medical device including a body comprising an expandable mesh and a valve configured to allow unidirectional flow of fluid out of an aneurysm.

FIG. 9 is a schematic and conceptual diagram illustrating a top-down view of the body of the system of FIG. 8.

FIG. 10 is a schematic and conceptual diagram illustrating another view of the valve of the system of FIG. 8.

FIG. 11 is a schematic diagram illustrating an example valve of the system of FIG. 8.

FIG. 12 is a schematic diagram illustrating another example valve of the system of FIG. 8.

FIG. 13 is a schematic diagram illustrating another example valve of the system of FIG. 8.

FIG. 14 is a schematic diagram illustrating another example valve of the system of FIG. 8.

FIG. 15 is a schematic diagram illustrating another example valve of the system of FIG. 8.

FIG. 16 is a schematic diagram illustrating an example aspiration system configured to control medical aspiration based on a cardiac cycle of a patient.

FIG. 17 is a flow diagram illustrating an example method of decompressing an aneurysm using a medical device including valve configured to allow unidirectional flow of fluid out of an aneurysm and an aspiration catheter.

### DETAILED DESCRIPTION

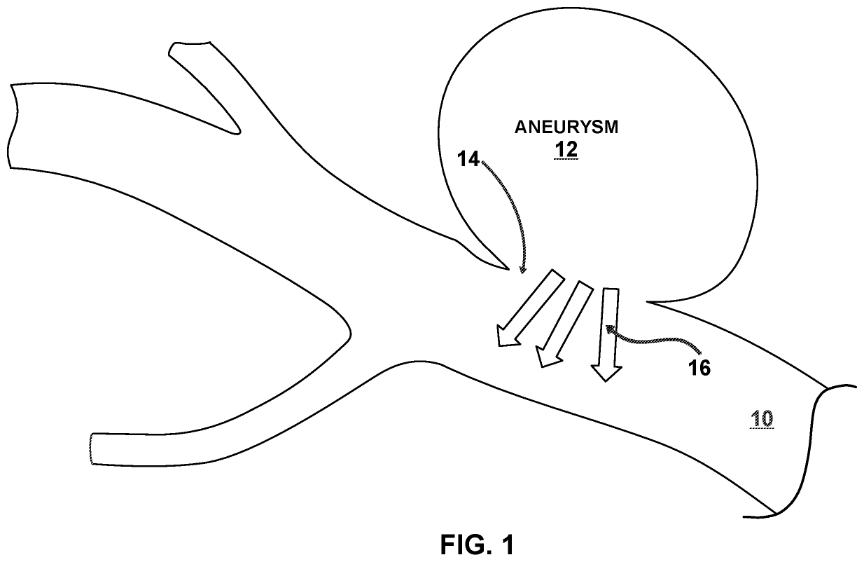

This disclosure describes devices, systems, and methods related to treating aneurysms. FIG. 1 is a conceptual diagram illustration of an example blood vessel 10 and an example aneurysm 12 formed in blood vessel 10. Aneurysm 12 may become weaker as blood flows from blood vessel 10 into aneurysm 12 through a neck 14 of aneurysm 12. Thus, blood flowing into aneurysm 12 may increase the risk of aneurysm rupture over time.

Current treatments or therapies for some aneurysms include endovascular coiling and liquid embolization. Endovascular coiling can help reduce or even stop the flow of blood into aneurysm 12 by placing a coil in aneurysm 12. The coil, which is left in a patient permanently, helps to occlude aneurysm 12, thus reducing or even preventing any future blood flow into aneurysm 12. Liquid embolization involves the delivery of liquid embolic agents, such as cyanoacrylate, into aneurysm 12 to help occlude aneurysm 12. Once the liquid embolic agent is delivered to into aneurysm 12 via a microcatheter and has made contact with blood, the agent precipitates and solidifies to occlude aneurysm 12. Both endovascular coiling and liquid embolic agents often result in no change in the size of aneurysm 12.

Other treatments or therapies for some aneurysms include flow diverters and surgical clips. Flow diverters involve stents that are placed across aneurysm neck 14, but can take on the order of months (e.g., two to six months) to occlude and reduce the size of aneurysm 12. Surgical clips help reduce blood flow into aneurysm 12, but can involve much more invasive procedures to place in a patient. Similar to endovascular coils and liquid embolic agents, flow diverters and surgical clips are often left in a patient permanently, with aneurysm 12 either never fully collapsing or collapsing over a prolonged period of time.

Example medical devices and systems described herein are configured to both occlude aneurysm 12 and reduce a size of aneurysm 12 relatively quickly, e.g., as compared to use of flow diverters and surgical clips alone. The example medical devices and systems are configured to be positioned over aneurysm neck 14 to facilitate unidirectional fluid flow 16 from inside aneurysm 12 and into vessel 10 to remove fluid from aneurysm 12. Removal of fluid from aneurysm 12 helps to reduce a size of aneurysm 12, and thus may also be referred to as decompression of aneurysm 12 herein. The medical devices and systems are also configured to divert blood flow from flowing from vessel 10 into aneurysm 12, which can facilitate healing of aneurysm 12. The decompression (e.g., collapsing) of aneurysm 12 may help reduce the risk of aneurysm rupture, reduce intracranial pressure more quickly than other treatments or therapies that take longer to act. Example medical devices and systems described herein can be used to treat aneurysms formed in any blood vessel in a patient. In some examples, the decompression of aneurysm 12 may be used to treat an aneurysm at the branching of an artery. A clinician can monitor the decompression of aneurysm 12 over time via fluoroscopy.

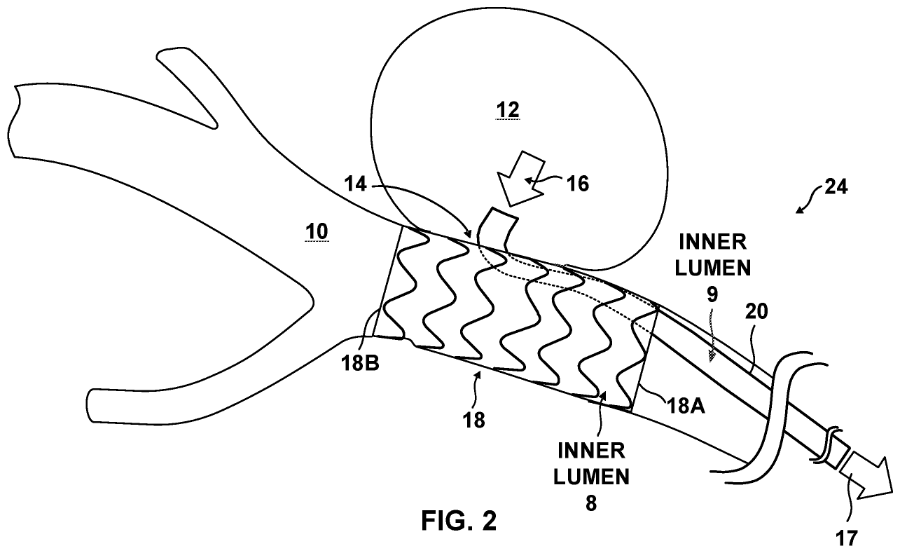

In some examples, medical aspiration is used to facilitate fluid flow 16 out of aneurysm 12 and into blood vessel 10 and decompress aneurysm 12 relatively quickly compared to examples in which no external forces are used to remove fluid from aneurysm 12. For example, a suction force (also referred to herein as suction, a vacuum force, or negative pressure) may be applied proximate the medical device and aneurysm neck 14. A suction source applies a suction force sufficient to create a negative pressure that draws a fluid, such as blood, an aspiration fluid, more solid material, or a mixture thereof, through a valve of the medical device out of aneurysm 12 and into blood vessel 10 and/or out of the body of the patient via a lumen of the aspiration catheter, as shown by arrow 17 in FIG. 2.

As used herein, “suction force” is intended to include within its scope related concepts such as suction pressure, vacuum force, vacuum pressure, negative pressure, fluid flow rate, and the like. A suction force can be generated by a vacuum, e.g. by creating a partial vacuum within a sealed volume fluidically connected to a catheter, or by direct displacement of liquid in a catheter or tubing via (e.g.) a peristaltic pump, or otherwise. Accordingly, suction forces or suction as specified herein can be measured, estimated, computed, etc. without need for direct sensing or measurement of force. A “higher,” “greater,” or “larger” (or “lower,” “lesser,” or “smaller”) suction force described herein may refer to the absolute value of the negative pressure generated by the suction source.

Once a medical device has been positioned over aneurysm neck 14, a clinician may apply the suction force via, for example, a catheter, and remove fluid from aneurysm 12 in the direction of fluid flow 16. The medical device is configured to enable fluid flow 16 out of aneurysm 12 to decompress or treat aneurysm 12 while also reducing or even preventing flow of fluid opposite to fluid flow 16, e.g., reduce or even prevent antegrade flow from blood vessel 10 into aneurysm 12. For example, the medical device can include a one-way valve configured to allow unidirectional fluid flow 16 through aneurysm neck 14 and to help prevent blood flow flowing back into aneurysm 12 via neck 14 (in a fluid flow direction opposite fluid flow 16). The fluid can flow directly through aneurysm neck 14 or through an opening of the medical device in examples in which the medical device is positioned in aneurysm neck 14. Once fluid has been removed from aneurysm 12, the medical device may remain implanted in the patient to help divert fluid flow from aneurysm 12 and provide long-term treatment of aneurysm 12. Thus, the medical device can be referred to as an endovascular implant.

A medical device described herein can have any suitable configuration. In examples described herein, the medical device is configured to be positioned across aneurysm neck 14 (e.g., from within aneurysm 12 and/or within blood vessel 10 outside of aneurysm 12) and includes a valve configured to enable unidirectional fluid flow 16 from aneurysm 12, through the valve, and into blood vessel 10.

In some examples, a medical device includes an expandable frame and polymer layer defining one or more flaps that act as a one-way valve configured to open towards a central longitudinal axis of the frame to allow the unidirectional flow of fluid out of aneurysm 12. In other examples, the medical device comprises a one-way valve defined by at least two overlapping polymer layers that are configured to enable fluid flow through a space between the at least two polymer layers. The overlapping polymer layers can act as a one-way valve when implanted in blood vessel 10 and oriented such that antegrade blood flow does not open and flow into the space between the polymer layers. In still other examples, the medical device comprises an expandable mesh configured to occlude at least a part of the neck of the aneurysm and a valve configured to allow unidirectional flow of fluid from the aneurysm into a vessel. The type of valve includes, but is not limited to, a spring check valve, a flap check valve, a plug check valve, or any combinations thereof. Additionally, in some examples, the type of valve includes a membrane valve comprising a membrane configured to allow unidirectional flow of fluid from the aneurysm into a vessel.

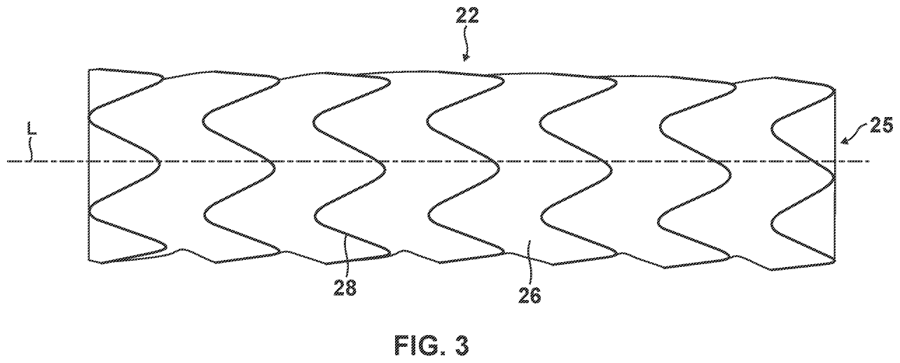

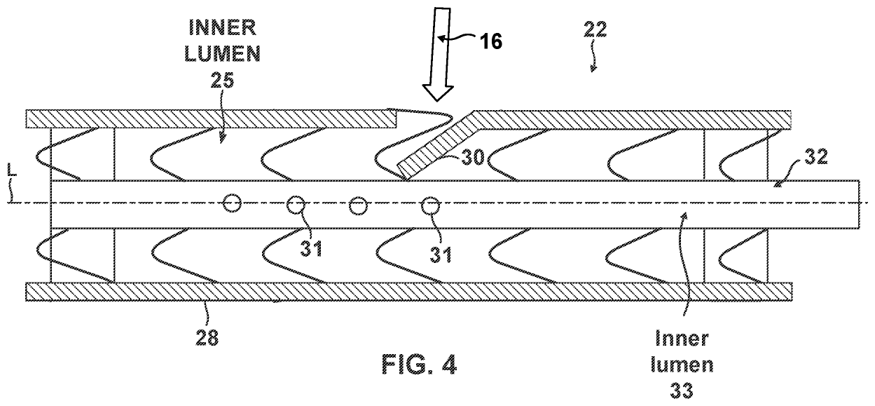

FIG. 2 is a schematic and conceptual diagram illustrating an example system 24 including an expandable member 18 and catheter 20, wherein a distal portion of catheter 20 is configured to extend through expandable member 18 and into aneurysm 12 through aneurysm neck 14. Expandable member 18 is an example of a medical device configured to be positioned across aneurysm neck 14 and including a valve configured to enable unidirectional fluid flow 16 (FIG. 1) out of aneurysm 12 and into blood vessel 10. In some examples, the valve is defined by slits or openings cut into a polymer layer or other membrane material covering expandable member 18, which is described in more detail with respect to FIG. 3 and FIG. 4 below.

Expandable member 18 is a device configured for long term (chronic) placement in vessel 10 to divert flow away from aneurysm 12. In the example of FIG. 2, expandable member 18 is configured to be positioned within vessel 10 and extend across neck 14 of aneurysm 12. That is, a length of expandable member 18 measured from a proximal end 18A of expandable member 18 to a distal end 18B of expandable member 18 is long enough to position the proximal end 18A proximal to neck 14 and the distal end 18B distal to neck 14. Expandable member 18 includes a valve configured to enable unidirectional fluid flow 16 out of aneurysm 12 through aneurysm neck 14 when expandable member 18 is positioned across aneurysm neck 14 and a suction force is applied via catheter 20 positioned in aneurysm 12 or in blood vessel 10 proximate aneurysm neck 14.

Expandable member 18 is configured to expand radially outwards from a relatively low-profile delivery configuration (also referred to herein as a compressed configuration) to the expanded configuration shown in FIG. 2. In some examples, expandable member 18 is configured to partially or fully self-expand from a collapsed configuration to an expanded configuration. For example, expandable member 18 may be formed from a shape-memory material (e.g., Nitinol) in a stent-like or mesh-like configuration. In other examples, expandable member 18 is not self-expandable and is configured to expanded with the aid of, for example, a balloon or another expandable structure.

In the expanded configuration, expandable member 18 is configured to engage with the inner wall of blood vessel 10 to secure expandable member 18 in place in blood vessel 10. For example, a radially outward force applied by expandable member 18 can be sufficient to hold expandable member 18 in apposition with an inner wall of blood vessel 10, thereby reducing or even preventing blood flow in the radial space between an outer surface of expandable member 18 and the inner wall of blood vessel 10. Expandable member 18 defines an inner lumen 8 that enables fluid to flow through expandable member 18, such that expandable member 18 does not occlude blood vessel 10. For example, expandable member 18 can be a stent or stent-like device.

Expandable member 18 defines an opening configured to be aligned with aneurysm neck 14 to enable fluid to flow out of aneurysm 12, through the opening, and into blood vessel 10. As discussed in further detail below with reference to FIGS. 3-5 and 8, in some examples, the valve of expandable member 18 can be positioned to enable one-way fluid flow out of aneurysm 12 via the opening, while minimizing or even preventing fluid flow back into aneurysm 12 from blood vessel 10 via the opening of expandable member 18.

In the example of FIG. 2, system 24 comprises catheter 20 that is configured to remove fluid from aneurysm 12 in the direction of fluid flow 16. Catheter 20 includes a relatively flexible elongated body configured to be navigated through vasculature of a patient. Catheter 20, or another catheter described herein, can be navigated through the vasculature and expandable member 18 until the distal tip of catheter 20 reaches aneurysm 12, such as in the example of FIG. 2. Expandable member 18 can help support efficient aspiration of aneurysm 12 via catheter by providing inner lumen 8 with a relatively large diameter for catheter 20 to engage with aneurysm 12.

A distal portion of catheter 20 is configured to extend through the valve of expandable member 18 and into aneurysm 12 through aneurysm neck 14. In some examples, catheter 20 extends through expandable member 18 into aneurysm 12 via an opening defined by expandable member 18. For example, expandable member 18 may have a frame-like structure defining a plurality of cells. Once catheter 20 is in its appropriate position, a suction force can be applied to an inner lumen 9 of catheter 20 and to aneurysm 12. More specifically, once a distal opening of catheter 20 is in aneurysm 12 or otherwise proximate aneurysm neck 14, a clinician may control a suction source to apply suction to remove a fluid from aneurysm 12 in the direction of fluid flow 16 through expandable member 18 until aneurysm 12 is sufficiently decompressed. In this way, system 24 of FIG. 2 facilitates the relatively quick decompression of aneurysm 12 compared to examples in which blood flows out of aneurysm 12 without the aid of presence of an external suction force.

In some examples, expandable member 18 is implanted across neck 14 with the aid of suction force. For example, a clinician may first deliver saline to vessel 10 to cause an unblocked aneurysm 12 expands and fills with saline. The clinician may then position expandable member 18 across aneurysm neck 14 and subsequently apply suction via catheter 20 such that aneurysm 12 is suctioned of the saline. Once under aspiration, expandable member 18 may then create a seal around aneurysm 12 in such a way that keeps aneurysm 12 evacuated even under blood pressure.

FIG. 3 is a more detailed schematic diagram of an example expandable member 22, which is an example of expandable member 18 of FIG. 2. As shown in FIG. 3, in some examples, expandable member 22 comprises polymer layer 26 and stent body 28. Polymer layer 26 comprises any suitable biocompatible material, such as, but not limited to, a polyester (e.g., a thermoplastic polymer resin such as Dacron available from DuPont de Nemours, Inc. of Wilmington, Delaware) or polytetrafluoroethylene (PTFE). In some examples, polymer layer 26 is blood impermeable. In these examples, polymer layer 26 defines one or more openings through which blood can be aspirated from aneurysm 12 through expandable member 22.

Stent body 28 may be a metal frame covered with polymer layer 26 (e.g., similar to a stent graft) on the inner or outer diameter of stent body 28 to block blood flow through the side wall of stent body 28. Stent body 28 can be radiopaque or can include one or more radiopaque markers to help a clinician identify the location of stent body 28 in the vasculature of a patient. Stent body 28 is configured to expand radially outward (e.g., via self-expansion or with the aid of an expansion device such as a balloon) within blood vessel 10 such that expandable member 22 contacts the inner wall of vessel 10 when it is in the expanded configuration.

Polymer layer 26 may be mechanically connected to (also referred to as attached in some examples) to stent body 28 via any suitable technique, such as, but not limited to, suturing, adhesion, ultrasonic welding, or thermal bonding. Polymer layer 26 can be connected to an inner and/or outer surface of stent body 28. In some examples, polymer layer 26 and stent body 28 of expandable member 22 are configured to create a seal over aneurysm neck 14.

In the example of FIG. 3, in the expanded configuration, expandable member 22 is configured to be in apposition with a wall of blood vessel 10 of a patient, e.g., expandable member 22 may contact the vessel wall to minimize or even prevent any fluid flow between the vessel wall and the exterior of expandable member 22. In this way, fluid flowing through vessel 10 is diverted through expandable member 22. As shown in FIG. 4 and described in further detail below, slits or openings may be cut into polymer layer 26 of expandable member 22, wherein the cut portions of polymer layer 26 can act as one-way flap valves that open when a negative pressure is applied inside expandable member 22 (such as in the presence of suction force applied by an aspiration catheter 20) and close in the presence of blood flow (e.g., antegrade blood flow) in blood vessel 10. Thus, as blood attempts to flow back into aneurysm 12 via aneurysm neck 14, the one-way flap valves may reduce or even prevent blood flow back into aneurysm 12. In some examples, the slits or openings may be cut throughout polymer layer 26 such that a sufficient number of slits or openings are present to allow fluid to flow out of aneurysm 12.

Stent body 28 can limit the maximum size of the slits or openings cut into polymer layer 26. For example, stent body 28 can include a plurality of interconnected struts that define a framework, and in order for the flap(s) to open (e.g., radially inward toward central longitudinal axis L of stent body 28), the length of the flap(s) may be limited by the space between adjacent struts. In some examples, the slits or openings defining the flap(s) are cut in way that allows the flaps to open radially inward toward central longitudinal axis L at an angle (e.g., as shown in FIG. 4, flap 30 opens radially inward toward central longitudinal axis L at a 45° angle from stent body 28). In some examples, the slits or openings defining the flap(s) are cut to allow the blood flow through blood vessel 10 and through inner lumen 25 of expandable member 22 to keep the flaps closed in the presence of antegrade blood flow through vessel 10 (from a proximal end of stent body 28 to a distal end of stent body 28). More specifically, polymer layer 26 may include one or more flaps configured to open towards a central longitudinal axis L of stent body 28 to allow the unidirectional flow of fluid out of aneurysm 12. The flaps may be strategically placed to align with parts of stent body 28 to enable stent body 28 to be used as support so that the flaps are unable to open outwards toward aneurysm 12, thus preventing antegrade flow of fluid into aneurysm 12.

When expandable member 22 is positioned in blood vessel 10 (FIG. 2) and spans across neck 14 (e.g., from a location proximal to neck 14 to a point distal to neck 14) of aneurysm 12 (FIG. 2), fluid flowing through vessel 10 flows through inner lumen 25 of expandable member 22. The flaps, also described herein as valves or flap valves, of polymer layer 26 helps reduce or even prevent fluid flowing through inner lumen 25 from entering aneurysm 12. Additionally, the flaps of polymer layer 26 are configured to allow fluid to flow out of aneurysm 12 through expandable member 22 and into vessel 10 in the direction of fluid flow 16. The flaps of polymer layer 26 are configured in such a way that results in the flaps to remain closed under blood pressure, e.g., remain closed as fluid flows through inner lumen 25 of expandable member 22. As fluid is able to flow out of aneurysm 12 through the flaps of polymer layer 26 and prevented from flowing in the opposite direction into aneurysm 12, system 24 in conjunction with expandable member 22 helps removes fluid to decompress aneurysm 12 and reduce or even prevent any future refilling or antegrade flow of fluid into aneurysm 12.

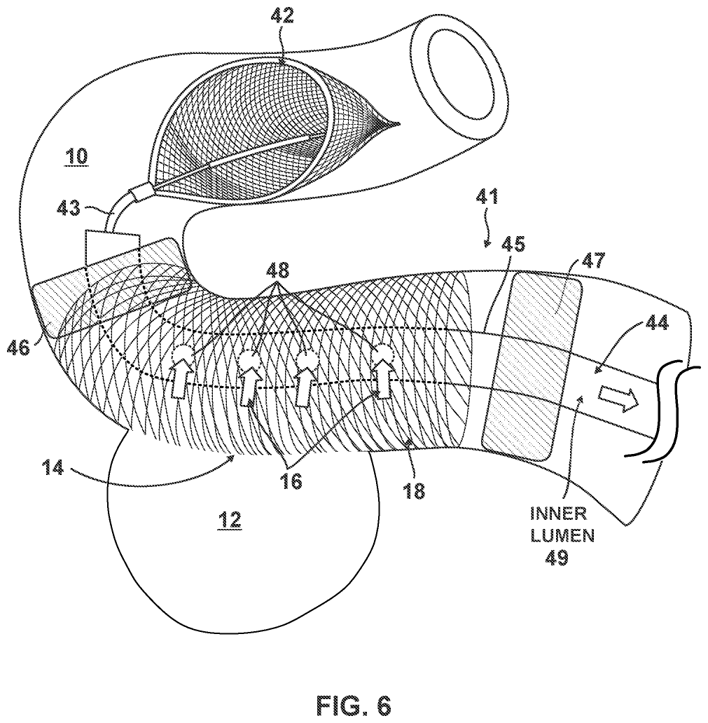

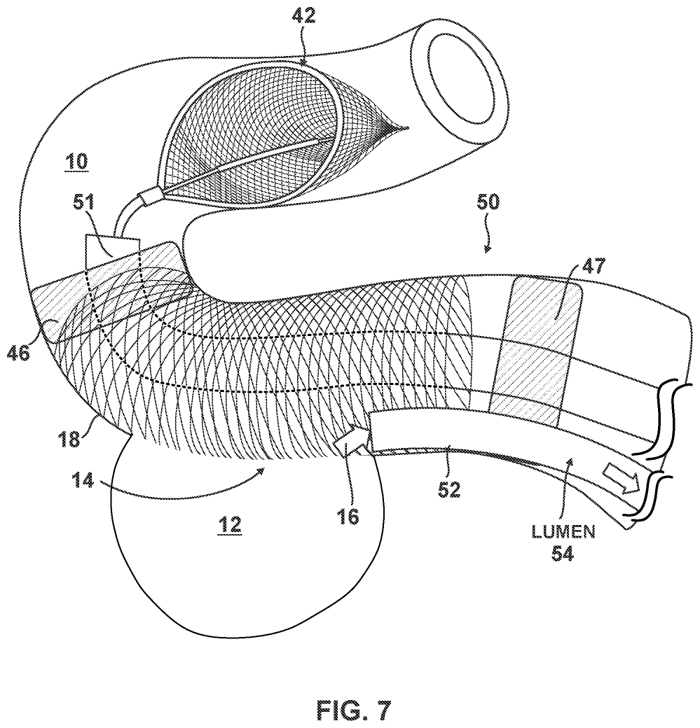

FIG. 4 is another schematic diagram of example expandable member 22 and example catheter 32, which are examples of expandable member 18 and catheter 20 of FIG. 2. In some examples, catheter 32 is a balloon guide catheter (including one, two, or more balloons), such as the examples of balloon guide catheter 44 in FIG. 6 or balloon guide catheter 51 in FIG. 7. FIG. 4 illustrates a flap 30 defined by polymer layer 26. Although one flap 30 is shown in FIG. 4, in other examples, polymer layer 26 can define any suitable number of flaps. As described with respect to FIG. 3, flap 30 functions as a one-way valve and is configured to open towards a central longitudinal axis L of stent body 28 to allow the unidirectional flow of fluid in the direction of fluid flow 16 out of aneurysm 12 to decompress aneurysm 12 and reduce or event prevent antegrade flow of fluid into aneurysm 12.

Catheter 32 defines an inner lumen 33 to which a suction source can apply a suction force to aspirate fluid from aneurysm 12 to facilitate a relatively quick decompression of aneurysm 12. The suction force can be applied proximate or within aneurysm 12 using any suitable openings in catheter 32. In the example shown in FIG. 4, catheter 32 includes a catheter body defining one or more side holes 31 open to inner lumen 33.

In the example of FIG. 4, catheter 32 is configured to remain in inner lumen 25 of expandable member 22 when fluid is aspirated from aneurysm 12. For example, during a medical procedure, a clinician can align side holes 31 with flap 30 to enable the suction force to draw fluid from aneurysm 12 into inner lumen 33 via side holes 31. The suction force can also cause flap 30 to open, even in the presence of blood flow through inner lumen 25. In these examples, rather than a distal portion of catheter 32 being configured to extend through a valve and into aneurysm 12 through aneurysm neck 14, catheter 32 is configured to be positioned proximate aneurysm neck 14 while remaining within blood vessel 10.

In contrast to catheters that are positioned in aneurysm 12 to aspirate fluid therefrom (e.g., through a distal opening of the catheter), with catheter 32 defining side holes 31, a clinician can be less precise in positioning catheter 32 relative to aneurysm neck 14 for an aspiration procedure. In contrast to catheter 20 shown in FIG. 2, catheter 32 is configured to apply suction force to aneurysm neck 14 from a position in blood vessel 10 (in a lumen of stent body 28).

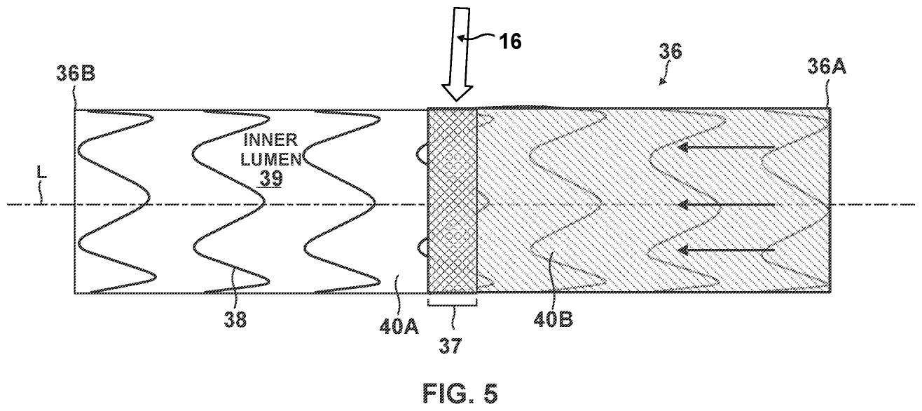

FIG. 5 is a schematic diagram of another example expandable member 36, which is another example of expandable member 18 of FIG. 2. Expandable member 36 includes stent body 38 and at least two overlapping polymer layers 40A, 40B (collectively, polymer layers 40) that are configured to enable fluid flow through a space between the at least two polymer layers 40A, 40B in the direction of fluid flow 16. Fluid flow 16 can, for example, represent antegrade blood flow that flows in a direction from a proximal end 36A of expandable member 36 to a distal end 36B of expandable member 36. Polymer layers 40A, 40B are arranged relative to each other such that fluid can flow from a location radially outwards of polymer layer 40A to a space between polymer layers 40A, 40B in the region of overlap 37 and into inner lumen 39 of expandable member 36. The space between polymer layers 40A, 40B can be, for example, radially inwards of polymer layer 40A and radially outwards of polymer layer 40B.

The at least two overlapping polymer layers 40 define a valve that is configured to allow unidirectional fluid flow 16 out of aneurysm 12 to help decompress aneurysm 12. An open state of the valve corresponds to a state in which portion of polymer layer 40B within the region of overlap 37 has moved towards central longitudinal axis L of stent body 38 away from polymer layer 40A and a closed state of the valve corresponds to a state in which a portion of polymer layer 40B within the region of overlap 37 remaining in its initial position directly adjacent to (e.g., in contact with) polymer layer 40A. In the region of overlap 37, polymer layer 40B is an inner polymer layer and polymer layer 40A is an outer polymer layer. The initial position of polymer layer 40B can be, for example, an at rest position in which no external suction forces are being applied from within inner lumen 39 of expandable member 36.

In the example of FIG. 5, the valve may remain closed under blood pressure and antegrade blood flow through blood vessel 10 and through inner lumen 39 and when outer polymer layer 40A is positioned in such a way that an opening or flap defined by the outer polymer layer remains covered by inner polymer layer 40B. Upon the application of the suction force, the opening or flap defined by the outer polymer layer becomes uncovered by the inner polymer layer, allowing fluid to flow from aneurysm 12 into inner lumen 39 of expandable member 36 in the direction of fluid flow 16, as previously described in the examples of FIG. 2 and FIG. 3.

In some examples, the two overlapping polymer layers 40 are not mechanically joined to each other. In other examples, the two overlapping polymer layers 40 are mechanically joined to each other, but still enable fluid flow through a radial space between the polymer layers 40.

Polymer layers 40 can be formed from any suitable number of layers, including, for example, two polymer layers 40A and 40B and from any suitable materials. Polymer layers 40 overlap by any suitable length, the length being measured along central longitudinal axis L of expandable member 36, and the length corresponding to the region of overlap 37. Polymer layers 40, for example, comprise of a 0.254 centimeter to 2.54 centimeters overlap. An overlap greater than 2.54 centimeters, for example, may cause suction to become more difficult. An overlap less than 0.245 centimeter, for example, may increase the risk of the valve not closing entirely.

As discussed above, in some examples, a medical system herein can include another medical device, such as an embolic protection element, configured to capture a thrombus (e.g., an emboli or other particles) that may move downstream of aneurysm 12 during a medical procedure to decompress aneurysm using any of the devices and systems described herein. The embolic protection element can include, for example, an occlusion member (e.g., a balloon or other expandable structure) and/or an embolic protection device (e.g., a filter). In some examples in which the embolic protection device is positioned distal (e.g., downstream in the direction of blood flow) to aneurysm 12, the embolic protection device can also be referred to as a distal protection device.

FIG. 6 is a schematic and conceptual diagram illustrating an example system 41 that includes expandable member 18, a distal embolic protection device 42, and a balloon guide catheter 44. A distal portion of balloon guide catheter 44 is configured to extend through expandable member 18 and includes an elongated catheter body 45 and a distal balloon 46 connected to or otherwise carried by catheter body 45. Distal balloon 46 is configured to be expanded in blood vessel 10 to occlude blood flow through vessel 10. This can help, for example, reduce or even prevent thrombi from flowing downstream of balloon guide catheter 44.

In some examples, in addition to or instead of distal balloon 46, balloon guide catheter 44 includes a proximal balloon 47. When balloon guide catheter 44 includes two balloons 46, 47, proximal balloon 47 is positioned proximal to distal balloon 46. Proximal balloon 47 can be, for example, configured to be positioned on an opposite side of side holes 48 defined by catheter body 45 from distal balloon 46. In the example of FIG. 6, balloons 46, 47 are on opposite sides of aneurysm 12. Expansion of proximal balloon 47 in blood vessel 10 helps to occlude blood flow through blood vessel 10 upstream of aneurysm 12, which may facilitate more efficient aspiration of fluid from aneurysm 12 at least by reducing further blood flow into aneurysm 12 during the aneurysm treatment procedure.

Balloons 46, 47 can be formed from any suitable material. In some examples, balloons 46, 47 are compliant balloons formed of material such as latex, silicon, a thermoplastic elastomer (e.g., Chronoprene available from AdvanSource Biomaterials of Wilmington, Massachusetts or Santoprene available from U.S. Plastic Corp of Lima, Ohio), or other elastomers and is positioned at or near the distal end of balloon guide catheter 44.

Catheter body 45 defines one or more side holes 48, which are open to inner lumen 49 defined by catheter body 45. Side holes 48, which are similar to side holes 31 of FIG. 4, define a fluid pathway for fluid flow from aneurysm 12 into inner lumen 49 (e.g., when a suction force is applied to inner lumen 49). Side holes 48 enable catheter body 45 to remain in blood vessel 10 during a medical aspiration procedure and facilitate decompression of aneurysm 12 without requiring catheter body 45 or any other part of balloon guide catheter 44 to be positioned inside aneurysm 12 and without requiring precise positioning of catheter body 45 relative to aneurysm 12. In the example of FIG. 6, balloon guide catheter 44 includes both balloons 46, 47 and includes side holes 48 for aspiration, thus eliminating the need for an additional microcatheter for aspiration. By enabling one balloon guide catheter 44 to be positioned for both vessel occlusion and aspiration, an overall procedure time can be reduced.

In the example of FIG. 6, balloon guide catheter 44 is configured to extend through the interior of expandable member 18. A clinician can position balloon guide catheter 44 in vessel 10 proximate aneurysm neck 14 and align the one or more side holes 48 with aneurysm neck 14 as well as the opening or valve defined by the body of expandable member 18. Expandable member 18 includes a valve configured to enable unidirectional fluid flow 16 out of aneurysm 12 through aneurysm neck 14 when expandable member 18 is positioned across aneurysm neck 14 and placed under the suction force applied by balloon guide catheter 44.

In the example shown in FIG. 6, side holes 48 are positioned between balloons 46, 47. When balloons 46, 47 are expanded in blood vessel 10 distal and proximal to aneurysm 12, respectively, and a suction force is applied to inner lumen 49, fluid that is in a volume between balloons 46, 47 is aspirated into inner lumen 49 via side holes 48, which can result in decompression of aneurysm 12. The expanded balloons 46, 47 may help increase the efficiency of the aneurysm decompression procedure by helping to direct the suction force to aneurysm 12. In addition, because balloons 46, 47, when expanded, reduce or even eliminate the blood flow between balloons 46, 47, balloons 46, 47 may reduce blood loss during the procedure by reducing the amount of blood that can be aspirated through inner lumen 9.

Distal embolic protection device 42 is configured to be introduced through inner lumen 49 of catheter body 45 to a position downstream of aneurysm 12. Distal embolic protection device 42 can be positioned on, for example, a distal portion of an elongated body 43 that has a length sufficient to extend from a location proximal to proximal end of catheter body 45, through inner lumen 49, and past a distal end of catheter body 45, as shown in FIG. 6. In addition, elongated body 43 has a relatively low profile so that it does not occupy a large portion of inner lumen 49 and enables a meaningful volume of fluid to be aspirated through inner lumen 49 even while elongated body 43 is positioned in inner lumen 49.

Distal embolic protection device 42 is a blood permeable filter in the example of FIG. 6, but can have other configurations (e.g., a blood impermeable device) in other examples. In examples in which a medical system includes distal embolic protection device 42, distal embolic protection device 42 can catch emboli or other material that flows downstream of aneurysm 12 during the aneurysm decompression procedure.

FIG. 7 is a schematic and conceptual diagram illustrating another example system 50 that includes distal embolic protection device 42. System 50 includes expandable member 18, balloon guide catheter 51, and aspiration catheter 52. Unlike balloon guide catheter 44 shown in FIG. 6, balloon guide catheter 51 does not include side openings or other openings intended for aspiration, but, rather, system 50 includes an aspiration catheter 52 that is separate from balloon guide catheter 51. The fluid in aneurysm 12 can be aspirated via aspiration catheter 52, which is separate from balloon guide catheter 51. A distal portion of balloon guide catheter 51 is configured to extend through expandable member 18, and a distal portion of aspiration catheter 52 is configured to extend through expandable member 18 and into aneurysm 12 through aneurysm neck 14, similar to the example of catheter 20 in FIG. 2.

In some examples, aspiration catheter 52 is a microcatheter configured to be positioned radially outwards of expandable member 18, e.g., between an inner wall of blood vessel 10 and an outer surface of expandable member 18. In some examples, aspiration catheter 52 extends through expandable member 18 into aneurysm 12 via an opening defined by expandable member 18. Once aspiration catheter 52 is in its appropriate position, a suction force can be applied to a lumen 54 of aspiration catheter 52 and to aneurysm 12. More specifically, once a distal opening of aspiration catheter 52 is positioned in aneurysm 12 or otherwise proximate aneurysm neck 14 (e.g., just outside of neck 14 in blood vessel 10), a clinician may suction fluid from aneurysm 12 in the direction of fluid flow 16 through expandable member 18 until the fluid in aneurysm 12 is decompressed.

As discussed above, in some examples, a medical device configured to help decompress and treat an aneurysm described herein can include an expandable body configured to be received in aneurysm 12 and occlude at least part of neck 14 of the aneurysm 12. For example, the expandable body can be formed at least partially from an expandable mesh that can be expanded within neck 14 to occlude at least part of neck 14 and extend into aneurysm 12 to also fill at least part of aneurysm 12. Further, the body may comprise a valve configured to allow unidirectional flow of fluid through the opening from aneurysm 12 into blood vessel 10 when the body is positioned across neck 14.

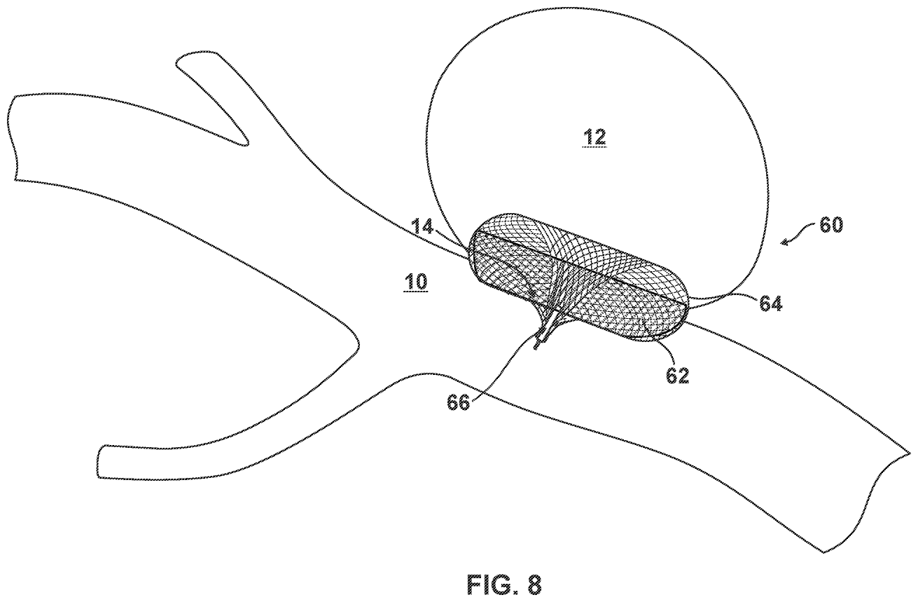

FIG. 8 is a schematic and conceptual diagram illustrating an example system 60 including expandable body 64 configured to be positioned across neck 14 of aneurysm 12, wherein expandable body 64 defines an opening configured to enable fluid flow out of aneurysm 12 through neck 14 when expandable body 64 is positioned across neck 14. Further, expandable body 64 is configured to extend into aneurysm 12 through neck 14 and expand to occlude at least part of neck 14 and at least part of aneurysm 12. Expandable body 64 is configured to expand radially outwards from a relatively low profile delivery configuration (e.g., compressed inside of a catheter) to an expanded configuration in which expandable body 64 has a maximum outer dimension (e.g., a diameter) larger than a maximum outer dimension of neck 14 of aneurysm 12. An example expanded configuration is shown in FIG. 8. The maximum outer dimension larger than neck 14 enables expandable body 64 to be anchored in aneurysm 12, as shown in FIG. 8.

Expandable body 64 can also be referred to as an expandable basket in some examples. In the example of FIG. 8, expandable body 64 is formed at least partially from or fully from an expandable mesh, the mesh being configured to occlude neck 14 of aneurysm 12. For example, a density of the mesh when expandable body 64 is in an expanded state may reduce or even prevent fluid flow through the mesh. In addition to or instead of having such a fluid impermeable expandable body 64, in some examples, at least part of expandable body 64 is covered with polymer layer 62. Polymer layer 62 is a fluid impermeable material to help reduce or even prevent fluid flow through expandable body 64, e.g., into aneurysm 12. Polymer layer 62 may be connected to expandable body 64 via any suitable technique, such as, but not limited to, suturing, adhesion, or thermal bonding. In other examples, expandable body 64 is not covered with a polymer layer.

In some examples, such as the example of FIG. 8, polymer layer 62 and expandable body 64 are configured to create a seal over aneurysm neck 14 to reduce or even prevent blood flow from blood vessel 10 into aneurysm. For example, expandable body 64 can be configured to be introduced through neck 14 and partially into aneurysm 12 while another part of expandable body 64 remains positioned in blood vessel 10, and then subsequently expanded such that expandable body 64 contacts the inner walls of aneurysm neck 14. Expandable body 64 may be secured relative to aneurysm neck 14 for relatively long-term treatment of aneurysm 12 using any suitable technique. In some examples, expandable body 64 applies a radially outward force to neck 14 sufficient to fix the position of expandable body 64 relative to neck 14. In addition to or instead of the radially outward force, expansion of expandable body 64 at least partially within aneurysm 12 can help anchor expandable body 64 within aneurysm 12 and reduce or even eliminate the possibility that expandable body 64 will be pulled back into blood vessel 10. In some examples, an adhesive, e.g., cyanoacrylate, is used to secure expandable body 64 relative to aneurysm neck 14. In some other examples, in addition to or instead of an adhesive, barbs, spikes, anchors, or the like extend from expandable body 64 to secure expandable body 64 relative to aneurysm neck 14.

Expandable body 64 also includes a one-way valve 66 that, as described in previous examples herein, is configured to allow unidirectional flow of fluid through aneurysm neck 14 into vessel 10 when expandable body 64 is positioned across aneurysm neck 14. Expandable body 64 includes an inner opening 68 that is large enough in diameter (or other cross-sectional dimension in the case of a non-circular opening) to accommodate one-way valve 66. In some examples, inner opening 68 extends through a portion of aneurysm neck 14, wherein the diameter of inner opening 68 is less than the diameter of aneurysm neck 14. In these examples, polymer layer 62 and expandable body 64 are still configured to create a seal over the portion of aneurysm neck 14 in which inner opening 68 does not extend, thus only allowing fluid to flow through inner opening 68 and one-way valve 66.

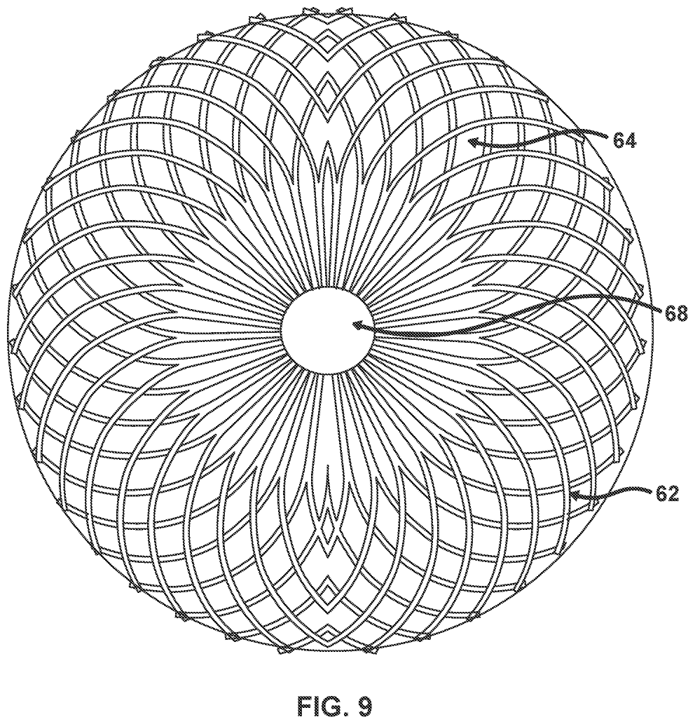

One-way valve 66 can have any suitable configuration that enables unidirectional fluid flow. For example, one-way valve 66 is configured to allow unidirectional flow of fluid through inner opening 68 of expandable body 64 from aneurysm 12 into vessel 10 when in an “open” position. Conversely, one-way valve 66, when in a “closed” position, is configured to prevent antegrade fluid flow through inner opening 68 of expandable body 64 into aneurysm 12. In some examples, one-way valve 66 includes, but is not limited to, a spring check valve, a flap check valve, a plug check valve, or any combination thereof. Additionally, in some examples, one-way valve 66 includes a membrane valve comprising a membrane configured to allow unidirectional flow of fluid from aneurysm 12 into vessel 10. Example one-way valves 66 are described with reference to FIGS. 9-13, but other one-way valves can be used in other examples.

In some examples, fluid from aneurysm 12 is suctioned through the lumen of a catheter that is also sued to deliver the expandable body 64 to aneurysm 12 or through a lumen of a separate catheter. For example, once the delivery catheter is in its appropriate position, a suction force can be applied to a lumen of the catheter and to aneurysm 12. More specifically, once a distal opening of a catheter is connected to expandable body 64 or otherwise proximate expandable body 64, the clinician may suction fluid from aneurysm 12 in the direction of fluid flow 16 through expandable body 64 until the fluid in aneurysm 12 is removed, e.g., system 60 of FIG. 8 enables the decompression or treatment of aneurysm 12.

FIG. 9 is a schematic and conceptual diagram illustrating a top-down view of expandable body 64 of FIG. 8. As shown in the example of FIG. 9, expandable body 64 includes polymer layer 62 and inner opening 68, which is configured to enable fluid flow out of aneurysm 12, through aneurysm neck 14, and into blood vessel 10. Inner opening 68 is configured to receive one-way valve 66, which defines a fluid pathway out of aneurysm 12 but helps reduce or even prevent fluid flow from blood vessel 10 into aneurysm 12. Thus, once aneurysm 12 is decompressed via aspiration of fluid out of aneurysm 12 through one-way valve 66, valve 66 may further help aneurysm 12 remain decompressed over time.

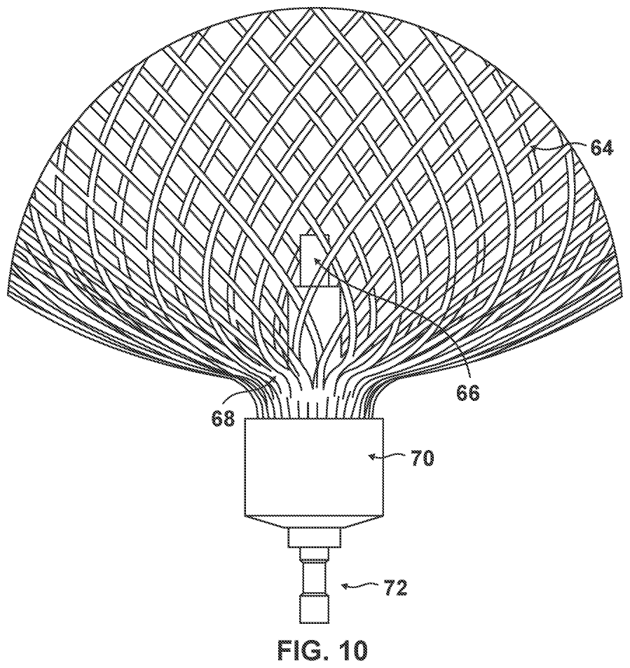

FIG. 10 is a schematic and conceptual diagram illustrating another view of expandable body 64 of FIG. 8. As shown in the example of FIG. 10, one-way valve 66 extends through inner opening 68 of expandable body 64 and is connected to expandable body 64 via band 70. In some examples, band 70 is connected to expandable body 64 via crimping, welding, adhesion, friction fit, or the like. In some examples, band 70 is a metal band. In some other examples, band 70 is a polymer band. In some examples, one-way valve 66 comprises delivery system 72 that extends and delivers fluid into blood vessel 10.

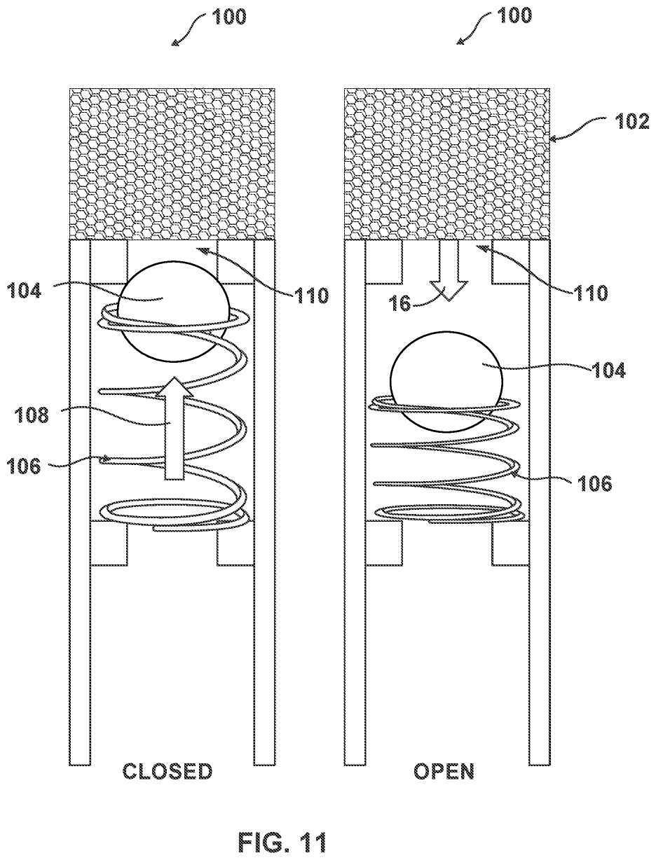

FIG. 11 is a schematic and conceptual diagram illustrating an example valve 100, which is an example of one-way valve 66 shown in FIG. 8. Valve 100, in the example of FIG. 11, is a spring check valve including a ball 104 and a spring 106. Ball 104 can be, for example, a rubberized ball that exhibits some elasticity. In some examples, mesh 102 of expandable body 64 acts as a filter and protects valve 100 from embolic blockages, while still enabling aspiration of fluid through valve 66. Valve 100 is configured to allow unidirectional flow of fluid through inner opening 68 of expandable body 64 from aneurysm 12 into vessel 10. That is, when valve 100 is in an “open” position, valve 100 defines a fluid passageway through which fluid can flow from aneurysm 12 in the direction of fluid flow 16. Valve 100, when in the “closed” position, prevents or reduces antegrade fluid flow 108 through inner opening 68 of expandable body 64 into aneurysm 12. The “open” position of valve 100, in this example, is defined by spring 106 being in a compressed position such that ball 104 does not occlude opening 110 of valve 100, wherein opening 110 is the fluid passageway through valve 100. The “closed” position of valve 100, in this example, is defined by spring 106 being in an extended position such that ball 104 occludes opening 110 of valve 100. In some examples, a clinician may use an aspiration catheter in conjunction with expandable body 64 including valve 100 to suction fluid from aneurysm 12 in the direction of fluid flow 16.

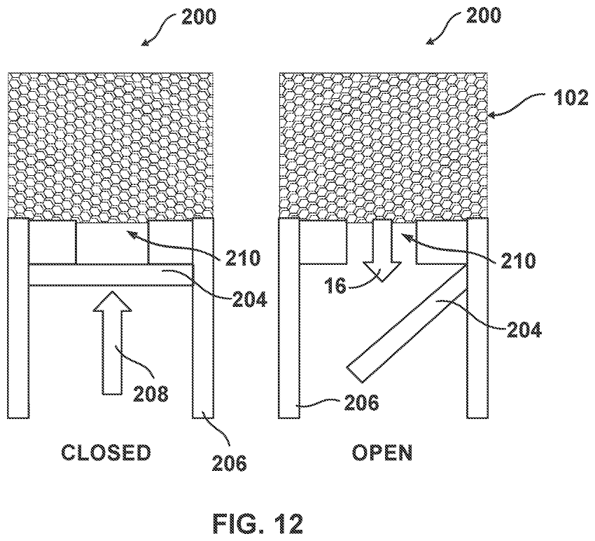

FIG. 12 is a schematic and conceptual diagram illustrating an example valve 200, which is another example of one-way valve 66 shown in FIG. 8. In the example of FIG. 12, valve 200 is a flap check valve including polymer flap 204, wherein polymer flap 204 is connected at one end to a valve body 206. In some examples, mesh 102 of expandable body 64 acts as a filter and protects valve 200 from embolic blockages. Valve 200 is configured to allow unidirectional flow of fluid through inner opening 68 of expandable body 64 from aneurysm 12 into vessel 10. That is, when valve 200 is in the “open” position, valve 200 defines a fluid passageway that allows fluid to flow from aneurysm 12 in the direction of fluid flow 16. Valve 200, when in the “closed” position, prevents or reduces antegrade fluid flow 208 through inner opening 68 of expandable body 64 into aneurysm 12. The “open” position of valve 200, in this example, is defined by polymer flap 204 being in a position such that polymer flap 204 does not occlude opening 210 of valve 200, wherein opening 210 is the fluid passageway through valve 200. The “closed” position of valve 200, in this example, is defined by polymer flap 204 being in a position such that polymer flap 204 occludes opening 210 of valve 200. In some examples, to a clinician may use an aspiration catheter in conjunction with expandable body 64 including valve 200 to suction fluid from aneurysm 12 in the direction of fluid flow 16.

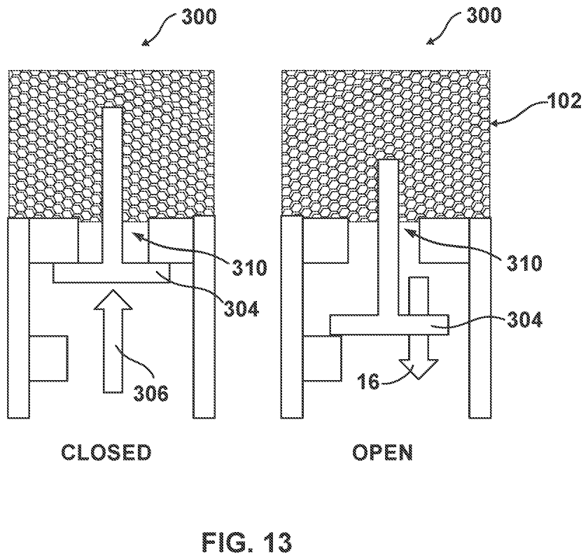

FIG. 13 is a schematic and conceptual diagram illustrating an example valve 300, which is another example of one-way valve 66 shown in FIG. 8. Valve 300, in the example of FIG. 13, is a plug check valve including plug and rod 304. In some examples, mesh 102 of expandable body 64 acts as a filter and protects valve 300 from embolic blockages. Valve 300 is configured to allow unidirectional flow of fluid through inner opening 68 of expandable body 64 from aneurysm 12 into vessel 10. That is, when valve 300 is in the “open” position, valve 300 defines a fluid passageway that allows fluid to flow from aneurysm 12 in the direction of fluid flow 16. Valve 300, when in the “closed” position, prevents or reduces antegrade fluid flow 306 through inner opening 68 of expandable body 64 into aneurysm 12. The “open” position of valve 300, in this example, is defined by plug and rod 304 being in a position such that plug and rod 304 does not occlude opening 310 of valve 300, wherein opening 310 is the fluid passageway through valve 300. The “closed” position of valve 300, in this example, is defined by plug and rod 304 being in a position such that plug and rod 304 occludes opening 310 of valve 300. In some examples, to a clinician may use an aspiration catheter in conjunction with expandable body 64 including valve 300 to suction fluid from aneurysm 12 in the direction of fluid flow 16.

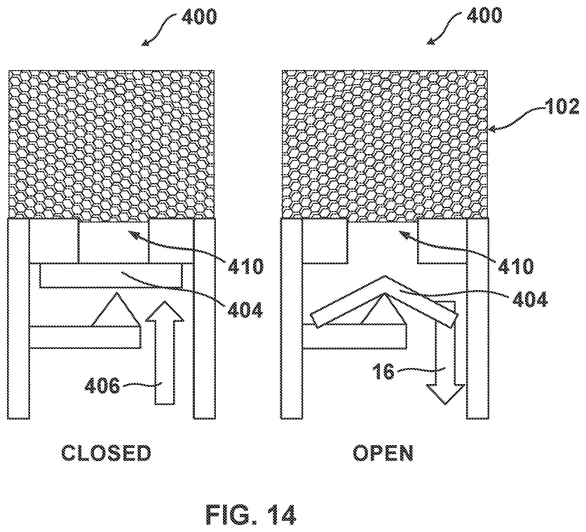

FIG. 14 is a schematic and conceptual diagram illustrating an example valve 400, which is another example of one-way valve 66 shown in FIG. 8. Valve 400, in the example of FIG. 14, is a plug check valve including membrane 404. In some examples, mesh 102 of expandable body 64 acts as a filter and protects valve 400 from embolic blockages. Valve 400 is configured to allow unidirectional flow of fluid through inner opening 68 of expandable body 64 from aneurysm 12 into vessel 10. That is, when valve 400 is in the “open” position, valve 400 defines a fluid passageway through which fluid can flow from aneurysm 12 in the direction of fluid flow 16. Valve 400, when in the “closed” position, prevents or reduces antegrade fluid flow 406 through inner opening 68 of expandable body 64 into aneurysm 12. The “open” position of valve 400, in this example, is defined by membrane 404 being in a position such that membrane 404 does not occlude opening 410 of valve 400, wherein opening 410 is the fluid passageway through valve 400. The “closed” position of valve 400, in this example, is defined by membrane 404 being in a position such that membrane 404 occludes opening 410 of valve 400. In some examples, to a clinician may use an aspiration catheter in conjunction with expandable body 64 including valve 400 to suction fluid from aneurysm 12 in the direction of fluid flow 16.

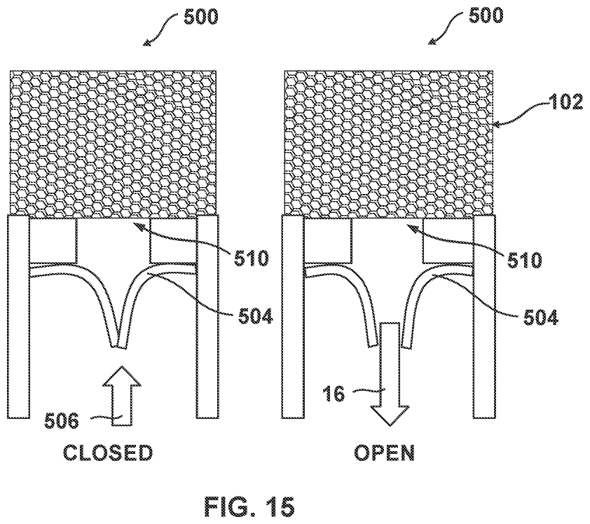

FIG. 15 is a schematic and conceptual diagram illustrating an example valve 500, which is another example of one-way valve 66 shown in FIG. 8. Valve 500, in the example of FIG. 15, is a heart-type valve including heart-type membrane 504. In some examples, the heart-type valve is a structure that includes leaflets configured to open and close (“coaptation into apposition”) opening 510 of the valve, e.g., the leaflets open in response to a force (e.g., a suction force from an aspiration catheter). In some examples, mesh 102 of expandable body 64 acts as a filter and protects valve 500 from embolic blockages. Valve 500 is configured to allow unidirectional flow of fluid through inner opening 68 of expandable body 64 from aneurysm 12 into vessel 10. That is, when valve 500 is in the “open” position, valve 500 defines a fluid passageway through which fluid can flow out of aneurysm 12 in the direction of fluid flow 16. Valve 500, when in the “closed” position, prevents or reduces antegrade fluid flow 506 through inner opening 68 of expandable body 64 into aneurysm 12. The “open” position of valve 500, in this example, is defined by heart-type membrane 504 being in a position such that heart-type membrane 504 does not occlude opening 510 of valve 500, wherein opening 510 is the fluid passageway through valve 500. The “closed” position of valve 500, in this example, is defined by heart-type membrane 504 being in a position such that heart-type membrane 504 occludes opening 510 of valve 500. In some examples, to a clinician may use an aspiration catheter in conjunction with expandable body 64 including valve 500 to suction fluid from aneurysm 12 in the direction of fluid flow 16.

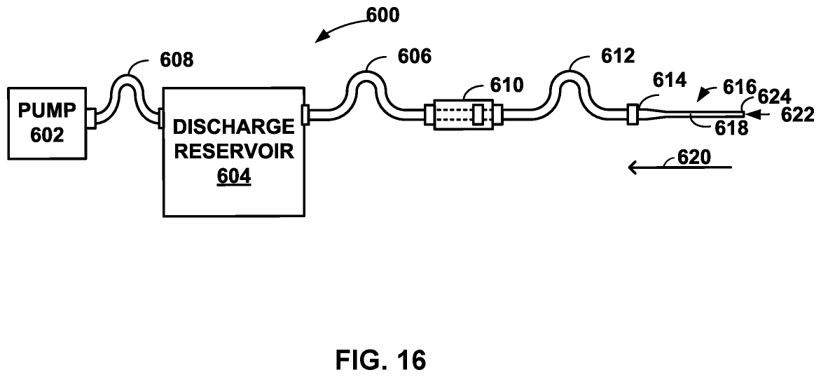

FIG. 16 is a schematic diagram illustrating an example aspiration system 600 that includes a catheter 616, a fluid flow switch 610 coupled to catheter 616 through aspiration tubing 612, and a pump 602. Aspiration system 600 may be used in various medical procedures, such as the decompression of an aneurysm. In some examples, catheter 616 may be substantially similar to example aspiration catheters described above, such as catheter 20 of FIG. 2, catheter 32 of FIG. 4, balloon guide catheter 44 of FIG. 6, or balloon guide catheter 51 of FIG. 7.

Aspiration system 600 is configured to remove fluid from catheter 616, e.g., draw fluid from catheter 616 into discharge reservoir 604, via a suction force applied by pump 602 to catheter 616 (e.g., to inner lumen 618 of catheter 616). For example, pump 602 can be configured to create a negative pressure within inner lumen 618 of catheter 616 to draw a fluid, such as blood, an aspiration fluid, more solid material, or a mixture thereof, in a direction indicated by arrow 620 and from aneurysm 12 and into inner lumen 618 via distal opening 622 of catheter 616. Distal opening 622, for example, can be placed inside aneurysm 12 through aneurysm neck 14, placed in blood vessel 10, or placed in blood vessel 10 proximate neck 14 without extending into aneurysm 12. The negative pressure within inner lumen 618 can create a pressure differential between inner lumen 618 and the environment external to at least a distal portion of catheter 616 that causes fluid and other material from the aneurysm to be introduced into inner lumen 618 via distal opening 622. For example, the fluid may flow from the aneurysm into inner lumen 618 via distal opening 622, and subsequently through aspiration tubing 612, fluid flow switch 610, and aspiration tubing 606 into discharge reservoir 604. Accordingly, the suction source of aspiration system 600 of FIG. 16 comprises pump 602, an evacuation volume in the form of discharge reservoir 604, and a pulsator in the form of fluid flow switch 610.

Catheter 616 and pump 602 can be fluidically coupled using any suitable configuration. In the example shown in FIG. 16, pump 602 is fluidically coupled to catheter 616 via aspiration tubing 608, discharge reservoir 604, aspiration tubing 606, fluid flow switch 610, and aspiration tubing 612. For example, pump 602 can be coupled to discharge reservoir 604 via aspiration tubing 608, and discharge reservoir 604 can be positioned between pump 602 and catheter 616. In these examples, pump 602 is configured to generate a partial vacuum in discharge reservoir 604 that causes fluid (e.g., blood) and more solid material (e.g., a thrombus) located within an inner lumen 618 of catheter 616 to be drawn into discharge reservoir 604 via tubing 612, 606 and fluid flow switch 610. In other examples, pump 602 can be more directly coupled to catheter 616 or may be further fluidically separated from catheter 616 by additional components.

Aspiration tubing 612, 606, 608, as well as other aspiration tubing described herein, is any suitable structure that defines a fluid pathway through which fluid and some relatively small fluid particles may flow between components of aspiration system 600. The tubing can be formed from any suitable material, such as, but not limited to, polymers, which can be reinforced with bonded, laminated or embedded tubular braids, coils, or other reinforcement member(s).

Catheter 616 is configured to be used as an aspiration catheter to remove fluid from an aneurysm of a patient. Catheter 616 defines at least one inner lumen, e.g., inner lumen 618 shown in FIG. 16, and at least one distal opening 622 that is open to inner lumen 618. Distal opening 622 may be at a distal-most end of catheter 616 and/or another position along catheter 616, such as in a sidewall of catheter 616 proximal to distal end 624 of catheter 616. For example, distal opening 622 may be substantially similar to side holes 31 in catheter 32 of FIG. 4 or side holes 48 in catheter body 45 of FIG. 6.

Catheter 616 includes an elongated body and a hub. The elongated body of catheter 616 is configured to be advanced through vasculature of a patient via a pushing force applied to a proximal portion of the elongated body. Catheter 616 includes any suitable construction for medical aspiration. In some examples, catheter 616 may include an inner liner, an outer jacket, and a structural support member, such as a coil and/or or a braid, positioned between at least a portion of the inner liner and at least a portion of the outer jacket. Catheter 616 may include other structures, such as an expandable member configured to radially expand within a vessel of a patient, e.g., to engage an aneurysm.

Catheter 616 is configured to be navigated to any aneurysm site in a patient. In some examples, catheter 616 is configured to access relatively distal locations in a patient including, for example, the middle cerebral artery (MCA), internal carotid artery (ICA), the Circle of Willis, and tissue sites more distal than the MCA, ICA, and the Circle of Willis. The MCA, as well as other vasculature in the brain or other relatively distal tissue sites (e.g., relative to the vascular access point), may be relatively difficult to reach with a catheter, due at least in part to the tortuous pathway (e.g., comprising relatively sharp twists or turns) through the vasculature to reach these tissue sites. The elongated body of catheter 616 may be structurally configured to be relatively flexible, pushable, and relatively kink- and buckle-resistant, so that it may resist buckling when a pushing force is applied to a relatively proximal section of catheter 616 to advance the elongated body distally through vasculature, and so that it may resist kinking when traversing around a tight turn in the vasculature. In some examples, the elongated body is configured to substantially conform to the curvature of the vasculature. In addition, in some examples, the elongated body has a column strength and flexibility that enables at least the distal portion of the elongated body to be navigated from a femoral artery, through the aorta of the patient, and into the intracranial vascular system of the patient, e.g., to reach a relatively distal treatment site. Alternatively, the elongated body can have a column strength (and/or be otherwise configured) to enable the distal portion of the elongated body to be navigated from a radial artery via an access site in the arm, e.g. at or near the wrist, through the aorta of the patient or otherwise to a common carotid or vertebral artery, and into the intracranial vascular system of the patient, e.g., to reach a relatively distal treatment site.

Although primarily described as being used to reach relatively distal vasculature sites, catheter 616 may also be configured to be used with other target tissue sites. For example, catheter 616 may be used to access tissue sites throughout the coronary and peripheral vasculature, the gastrointestinal tract, the urethra, ureters, fallopian tubes, veins and other body lumens. A length of catheter 616 may depend on the location of the target tissue site within the body of a patient or may depend on the medical procedure for which catheter 616 is used.

Pump 602 is configured to create a negative pressure (e.g., vacuum or suction) or otherwise induce fluid flow in inner lumen 618 of catheter 616, e.g., to draw fluid from an aneurysm through inner lumen 618 and into discharge reservoir 604. Thus, pump 602 is configured to generate a pressure differential that causes fluid in inner lumen 618 to be drawn out of inner lumen 618 and towards pump 602, e.g., into discharge reservoir 604. For example, pump 602 may include a port configured to couple to aspiration tubing 608, such that the negative pressure created by fluid pump 602 may be applied to the port and through aspiration tubing 608 to a fluid pathway between aspiration tubing 608 and inner lumen 618 of catheter 616. In the example shown in FIG. 16, the fluid pathway further includes discharge reservoir 604, aspiration tubing 612, 606, and fluid flow switch 610. In an example operation of pump 602, when distal opening 622 of catheter 616 is not blocked, pump 602 may draw fluid from inner lumen 618 of catheter 616 into discharge reservoir 604 through aspiration tubing 612, 606, and through fluid flow switch 610. As another example, when distal opening 622 is partially or wholly blocked, pump 602 may draw fluid from catheter 616 at a reduced flow rate or, in some instances in which blockage is complete, draw no fluid at all. However, even when distal opening 622 is blocked, pump 602 may be configured to continue to create a vacuum on inner lumen 618 of catheter 616, e.g. via further evacuation of air from discharge reservoir 604.

Pump 602 may also be referred to as a fluid pump and can have any suitable configuration. For example, pump 602 (as well as pumps generally within the present disclosure) can include one or more of a positive displacement pump (e.g., a peristaltic pump, a rotary pump, a reciprocating pump, or a linear pump), a centrifugal pump, and the like. In some examples, pump 602 includes a motor driven pump, while in other examples, pump 602 can include a syringe configured to be controlled by control circuitry, and mechanical elements such as linear actuators, stepper motors, etc. As further examples, the pump 602 could comprise a water aspiration venturi or ejector jet.

In some examples, pump 602 may be configured for bi-directional operation. For example, pump 602 may be configured to create a negative pressure that draws fluid from inner lumen 618 of catheter 616 in a first flow direction and create a positive pressure that pumps fluid to catheter 616 and through inner lumen 618 of catheter 616 in a second, opposite flow direction. As an example of this bi-directional operation, an operator of aspiration system 600 may operate pump 602 to pump an aspiration/irrigating fluid, such as saline, from an aspiration fluid reservoir (not shown in FIG. 16) to flush and/or prime catheter 616 (e.g., an infusion state) and subsequently draw fluid from a site of distal opening 622 of catheter 616, such as saline and/or blood, into discharge reservoir 604.

In some examples, aspiration system 600 includes fluid flow switch 610 (also referred to herein as a fluid switch) to control fluid flow through aspiration system 600. Fluid switch 610 may be configured to start and stop fluid flow from catheter 616 toward pump 602 (or in the opposite direction). For example, fluid switch 610 may have an “open” position corresponding to flow of fluid through fluid switch 610 and a “closed” position corresponding to no flow of fluid through fluid switch 610. A variety of switching mechanisms may be used for fluid switch 610 including, but not limited to, valves, sliders, clamps and the like. In some examples, fluid switch 610 may be configured for unaided operation by a clinician. For example, a mechanism of blocking fluid flow through fluid switch 610 may be directly operable by a mechanical force provided by the clinician. In some examples, fluid switch 610 is an automatic switch. In these examples, aspiration system 600 monitors pressure within the system, and in response to a “high” pressure reading, e.g., when aneurysm 12 has been fully evacuated or aspiration system 600 is clogged, aspiration system 600 automatically forces fluid switch 610 to a closed position. Such control of fluid switch 610 can be performed by control circuitry of aspiration system 600 or via a more mechanical construction of a fluid switch that closes in response to a threshold pressure or a threshold change in pressure. In other examples, system 600 does not include fluid switch 610.

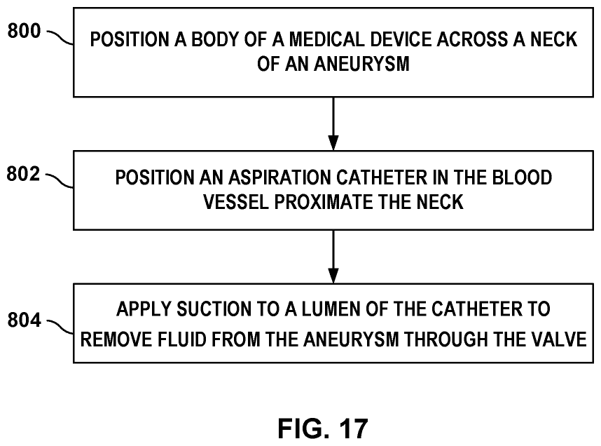

FIG. 17 is a flow diagram illustrating an example method of decompressing an aneurysm using a medical device including a valve configured to allow unidirectional flow of fluid out of an aneurysm and an aspiration catheter. The technique of FIG. 17 first includes positioning a body of a medical device across neck 14 of aneurysm 12, wherein the body defines an opening configured to enable fluid flow 16 out of aneurysm 12 through neck 14 when the body is positioned across neck 14. (800). The medical device may be, for example, to expandable members 18, 22, and 36 of FIGS. 2, 3, and 5, respectively, and/or expandable body 64 of FIG. 8. The valve may be, for example, flap 30 of FIG. 4 and/or valves 100, 200, 300, 400, and 500 of FIGS. 11, 12, 13, 14, and 15, respectively.

The technique of FIG. 17 further includes positioning an aspiration catheter in the blood vessel proximate the neck (802) and applying suction to the inner lumen to remove fluid from aneurysm 12 through the valve (804) and decompress aneurysm 12. The aspiration catheter may be, for example, any one or more of catheters 20, 32, 48, and 52 of FIGS. 2, 4, 6, and 7, respectively. Once the suction force is removed, the valve of the medical device can reduce or even prevent fluid flow back into aneurysm 12.

The disclosure includes the following examples. The examples described herein may be combined in any permutation or combination.
Example 1: A medical system including a medical device configured to direct fluid flow away from an aneurysm, the medical device including a body configured to be positioned across a neck of an aneurysm, the body defining an opening configured to enable fluid flow out of the aneurysm through the neck when the body is positioned across the neck; and a valve configured to allow unidirectional flow of fluid through the opening from the aneurysm into a vessel when the body is positioned across the neck.
Example 2: The medical system of example 1, further comprising a polymer layer defining one or more flaps, wherein the valve includes the one or more flaps defined by the polymer layer.
Example 3: The medical system of example 2, wherein the body comprises an expandable frame, and wherein the polymer layer is connected to the frame, and wherein the one or more flaps defined by the polymer layer are configured to open towards a central longitudinal axis of the frame to allow the unidirectional flow of fluid out of the aneurysm.
Example 4: The medical system of example 3, wherein the expandable frame and the polymer layer are configured to create a seal over the neck of the aneurysm when the one or more flaps are in a closed position.
Example 5: The medical system of any of examples 1-4, wherein the body comprises an expandable frame, and wherein the medical device further comprises at least two overlapping polymer layers connected to the frame, wherein the at least two overlapping polymer layers are configured to enable fluid flow through a space between the at least two polymer layers, and wherein the valve is defined by the at least two overlapping polymer layers.
Example 6: The medical system of any of examples 1-4, wherein the body comprises an expandable mesh, at least part of the mesh being is configured to be received in the aneurysm, and wherein the mesh is configured to occlude at least part of the neck of the aneurysm.
Example 7: The medical system of example 6, wherein the valve includes a spring check valve.
Example 8: The medical system of example 6, wherein the valve includes a flap check valve.
Example 9: The medical system of example 6, wherein the valve includes a plug check valve.
Example 10: The medical system of example 6, wherein the valve includes a membrane valve comprising a membrane configured to allow unidirectional flow of fluid through the opening.
Example 11: The medical system of any of examples 1-10, further including a catheter defining a catheter lumen, the catheter being configured to be positioned in the vessel; and a suction source configured to apply a suction force to the catheter lumen to remove fluid from the aneurysm through the valve and through the opening.
Example 12: The medical system of example 11, wherein the catheter defines one or more side holes configured to be aligned with the opening, wherein the one or more side holes facilitate fluid flow into the catheter lumen.
Example 13: The medical system of example 11, wherein a distal portion of the catheter is configured to extend through the valve and into the aneurysm through the neck of the aneurysm.
Example 14: A method including positioning a body of a medical device across a neck of an aneurysm, the body defining an opening configured to enable fluid flow out of the aneurysm through the neck when the body is positioned across the neck, the medical device further comprising a valve configured to allow unidirectional flow of fluid through the opening from the aneurysm into a blood vessel; and positioning a catheter in the blood vessel proximate the neck, the catheter defining a catheter lumen; and applying suction to the catheter lumen to remove fluid from the aneurysm through the valve.
Example 15: The method of example 14, wherein the catheter defines one or more side holes open to the catheter lumen, and wherein positioning catheter in the blood vessel proximate the neck comprises aligning the side holes with the neck, and wherein applying suction to the catheter lumen to remove fluid from the aneurysm through the valve comprises applying suction to the catheter lumen to remove fluid from the aneurysm through the valve and into the catheter lumen through the one or more side holes.
Example 16: The method of example 14 or example 15, further comprising introducing an expandable mesh in the blood vessel, wherein at least part of the mesh is configured to be received in the aneurysm, wherein the mesh is configured to occlude at least part of the neck of the aneurysm, and wherein the valve is positioned within the expandable mesh to enable fluid flow out the aneurysm and into the blood vessel.
Example 17: A medical system includes an intravascular medical device configured to be positioned across a neck of an aneurysm, the medical device comprising a one-way valve configured to allow flow of fluid out of the aneurysm and into a vessel when the medical device is positioned across the neck; and an aspiration catheter defining a distal opening configured to be positioned proximate the one-way valve to apply a suction force that facilitates the flow of fluid out of the aneurysm through the one-way valve.
Example 18: The medical system of example 17, wherein the aspiration catheter defines a catheter lumen, the medical system further includes a suction source configured to apply the suction force to the catheter lumen to remove fluid from the aneurysm through the one-way valve and into the catheter lumen via the distal opening.
Example 19: The medical system of example 17 or example 18, wherein the intravascular medical device comprises: an expandable frame; and a polymer layer connected to the expandable frame, the polymer layer defining one or more flaps, wherein the one-way valve includes the one or more flaps defined by the polymer layer.
Example 20: The medical system of example 17 or example 18, wherein the intravascular medical device comprises: an expandable frame; and at least two overlapping polymer layers connected to the expandable frame, wherein the at least two overlapping polymer layers are configured to enable fluid flow through a space between the at least two polymer layers, and wherein the one-way valve is defined by the at least two overlapping polymer layers.
Example 21: The medical system of example 17 or example 18, wherein the intravascular medical device comprises an expandable mesh configured to occlude at least part of the neck of the aneurysm, and wherein at least part of the mesh being is configured to be received in the aneurysm.
Example 22: The medical system of any of examples 17-21, wherein the one-way valve includes at least one of a spring check valve, a flap check valve, a plug check valve, or a membrane valve.

Various examples of the disclosure have been described. Any combination of the described systems, operations, or functions is contemplated. These and other examples are within the scope of the following claims.

What is claimed is:

1. A medical system comprising:
a medical device configured to direct fluid flow away from an aneurysm, the medical device comprising:
a body configured to be positioned across a neck of an aneurysm, the body defining an opening configured to enable fluid flow out of the aneurysm through the neck when the body is positioned across the neck; and
a valve configured to allow unidirectional flow of fluid through the opening from the aneurysm into a vessel when the body is positioned across the neck.

2. The medical system of claim 1, further comprising a polymer layer defining one or more flaps, wherein the valve includes the one or more flaps defined by the polymer layer.

3. The medical system of claim 2, wherein the body comprises an expandable frame, and wherein the polymer layer is connected to the frame, and wherein the one or more flaps defined by the polymer layer are configured to open towards a central longitudinal axis of the frame to allow the unidirectional flow of fluid out of the aneurysm.

4. The medical system of claim 3, wherein the expandable frame and the polymer layer are configured to create a seal over the neck of the aneurysm when the one or more flaps are in a closed position.

5. The medical system of claim 1, wherein the body comprises an expandable frame, and wherein the medical device further comprises at least two overlapping polymer layers connected to the frame, wherein the at least two overlapping polymer layers are configured to enable fluid flow through a space between the at least two polymer layers, and wherein the valve is defined by the at least two overlapping polymer layers.

6. The medical system of claim 1, wherein the body comprises an expandable mesh, at least part of the mesh being is configured to be received in the aneurysm, and wherein the mesh is configured to occlude at least part of the neck of the aneurysm.

7. The medical system of claim 6, wherein the valve includes at least one of a spring check valve, a flap check valve, or a plug check valve.

8. The medical system of claim 6, wherein the valve includes a membrane valve comprising a membrane configured to allow unidirectional flow of fluid through the opening.

9. The medical system of claim 1, further comprising:
a catheter defining a catheter lumen, the catheter being configured to be positioned in the vessel; and
a suction source configured to apply a suction force to the catheter lumen to remove fluid from the aneurysm through the valve and through the opening.

10. The medical system of claim 9, wherein the catheter defines one or more side holes configured to be aligned with the opening, wherein the one or more side holes facilitate fluid flow into the catheter lumen.

11. The medical system of claim 9, wherein a distal portion of the catheter is configured to extend through the valve and into the aneurysm through the neck of the aneurysm.

12. A method comprising:
positioning a body of a medical device across a neck of an aneurysm, the body defining an opening configured to enable fluid flow out of the aneurysm through the neck when the body is positioned across the neck, the medical device further comprising a valve configured to allow unidirectional flow of fluid through the opening from the aneurysm into a blood vessel; and
positioning a catheter in the blood vessel proximate the neck, the catheter defining a catheter lumen; and
applying suction to the catheter lumen to remove fluid from the aneurysm through the valve.

13. The method of claim 12, wherein the catheter defines one or more side holes open to the catheter lumen, and wherein positioning catheter in the blood vessel proximate the neck comprises aligning the side holes with the neck, and wherein applying suction to the catheter lumen to remove fluid from the aneurysm through the valve comprises applying suction to the catheter lumen to remove fluid from the aneurysm through the valve and into the catheter lumen through the one or more side holes.

14. The method of claim 12, further comprising introducing an expandable mesh in the blood vessel, wherein at least part of the mesh is configured to be received in the aneurysm, wherein the mesh is configured to occlude at least part of the neck of the aneurysm, and wherein the valve is positioned within the expandable mesh to enable fluid flow out the aneurysm and into the blood vessel.

15. A medical system comprising:
an intravascular medical device configured to be positioned across a neck of an aneurysm, the medical device comprising a one-way valve configured to allow flow of fluid out of the aneurysm and into a vessel when the medical device is positioned across the neck; and
an aspiration catheter defining a distal opening configured to be positioned proximate the one-way valve to apply a suction force that facilitates the flow of fluid out of the aneurysm through the one-way valve.

16. The medical system of claim 15, wherein the aspiration catheter defines a catheter lumen, the medical system further comprising:
a suction source configured to apply the suction force to the catheter lumen to remove fluid from the aneurysm through the one-way valve and into the catheter lumen via the distal opening.

17. The medical system of claim 15, wherein the intravascular medical device comprises:
an expandable frame; and
a polymer layer connected to the expandable frame, the polymer layer defining one or more flaps, wherein the one-way valve includes the one or more flaps defined by the polymer layer.

18. The medical system of claim 15, wherein the intravascular medical device comprises:
an expandable frame; and
at least two overlapping polymer layers connected to the expandable frame, wherein the at least two overlapping polymer layers are configured to enable fluid flow through a space between the at least two polymer layers, and wherein the one-way valve is defined by the at least two overlapping polymer layers.

19. The medical system of claim 15, wherein the intravascular medical device comprises an expandable mesh configured to occlude at least part of the neck of the aneurysm, and wherein at least part of the mesh being is configured to be received in the aneurysm.

20. The medical system of claim 15, wherein the one-way valve includes at least one of a spring check valve, a flap check valve, a plug check valve, or a membrane valve.
